(19) United States
(12) Patent Application Publication
(10) Pub. No.: US 2008/0193412 A1
(43) Pub. Date: Aug. 14, 2008

(54) Method of Enhancing the Immune Response to a Vaccine

(75) Inventor: Michael Tovey, Paris (FR)

Correspondence Address: BROWDY AND NEIMARK, P.L.L.C.;624 NINTH STREET, NW, SUITE 300, WASHINGTON, DC, 20001-5303, US

(73) Assignee: PHARMA PACIFIC PTY LTD., Laverton North (AU)

(21) Appl. No.: 11/629,076

(22) PCT Filed: Jun. 13, 2005
(86) PCT No.: PCT/US2005/020500

§ 371 Date: Dec. 27, 2007

Related U.S. Application Data
(60) Provisional application No. 60/579,186, filed on Jun. 12, 2004.

(51) Int. Cl. A61K 38/21; A61K 39/00; A61K 39/145; A61K 39/275; A61K 39/29

(52) U.S. Cl. 424/85.5; 424/85.4; 424/184.1; 424/209.1; 424/232.1; 424/227.1; 424/204.1; 424/231.1

(57) ABSTRACT

A method for enhancing the immune response to an antigen or vaccine comprising administering an effective amount of a Th1 stimulatory cytokine, preferably interferon, oromucosally at substantially the same time as administration of an effective amount of an antigen or vaccine.

### BACKGROUND OF THE INVENTION

1. Field of the Invention

The present invention relates to a method for enhancing the immune response to vaccination. More particularly, it relates to the use of interferon, and/or other Th1 stimulatory cytokines, as in an immunoadjuvant for enhancing the immune response following administration of a vaccine.

2. Description of the Related Art

Vaccines are known in the art. In general, they include killed or attenuated pathogens and subunit vaccines, or vaccines comprising another antigen against which an immune response is desired, which are administered with the aim of preventing, ameliorating or treating infectious diseases.

In particular, subunit vaccines are vaccines based on antigens derived from components of the pathogen that are considered to be important targets for protection mediated by the host's immune system. Although proved to be highly safe, subunit vaccines often induce inadequate immune responses due to the fact that the antigen upon which they are based is either poorly immunogenic or nonimmunogenic.

Hence, in order to raise immunogenicity, subunit vaccines often need to include or be administered together with an adjuvant. Adjuvants are defined with respect to immunology, as “a vehicle used to enhance antigenicity” (Stedman's Medical Dictionary, 2003). Thus, an adjuvant is a substance that, when administered together with the antigen, enhances the antigenicity thereof as compared with the antigen alone.

Although many types of adjuvants have been used in animal models and classical examples include water-in-oil emulsions in which antigen solution is emulsified in mineral oil (Freund's incomplete adjuvant), sometimes with the inclusion of killed mycobacteria (Freund's complete adjuvant), a suspension of minerals (alum, aluminum hydroxide or phosphate) on which antigen is adsorbed, saponins and LPS-derived products, currently, aluminum-based mineral salts are the only adjuvants routinely included in the vaccine formulations in humans. Although safe, such salts are weak adjuvants for antibody induction and are not capable of stimulating classical cell-mediated immune responses.

Induction of both antibodies and a cell-mediated response is required to provide a highly effective defense against invading pathogens with the aim of limiting their spread or eliminating them. Vaccines need to provide or induce two types of signals in order to elicit a strong, protective immune response. Firstly, vaccines need to deliver the antigen, which triggers antigen-specific receptors on T and B lymphocytes. Secondly, effective vaccines need to induce the expression of co-stimulatory molecules by antigen presenting cells, which then promote a strong response by the antigen-triggered lymphocytes. This second signal is often provided by factors associated with infection, when using vaccines containing live pathogens, but is generally lacking in subunit vaccines, resulting in their poor immunogenicity. The addition of an adjuvant that can contribute this second signal will enhance the effectiveness of the vaccine and, further, may dictate the type of immune response elicited.

These signals direct the host's immune system towards the subsequent development of effector mechanisms that characterize the type and potency of the overall immune response to a given infectious agent.

Cytokines represent the major factors involved in the communication between immunocompetent cells, including T cells, B cells, macrophages, and dendritic cells, during the course of an immune response to antigens and infectious agents. A number of studies on mouse and human T helper (Th) clones has provided extensive evidence for the existence of different activities exhibited by Th cells (called Th1 and Th2), which was inferred from the profile of cytokine secretion. Thus, production of IFN-γ or IL-4 are considered as the typical hallmarks of a Th1 or Th2 response, respectively. The Th1 type of immune response is generally associated with IgG2a production in mice and the development of cellular immunity, whereas the Th2 type of response is associated with IgE production, eosinophils and mast cell production. It is generally thought that induction of a Th1 type of immune response is instrumental in the generation of a protective immune response to viruses and certain bacterial infections. In this regard, it is important to note that clinically available adjuvants, such as aluminum-based mineral salts, tend to induce a Th2 type of immune response, which can cause an allergic response that contributes to their undesirable side effects.

Influenza vaccination reduces morbidity and mortality caused by influenza infection in high risk groups including the aged and individuals with an impaired immune response, but is not totally protective in all recipients (Oxford et al., 2003). The protection provided by the commonly used subunit vaccines is thought to be principally due to the production of antibodies to viral hemagglutinin, as such vaccines elicit a poor cytotoxic T-cell response, and the hemagglutination inhibitory (HI) antibody titer is generally used as a surrogate marker of protection. Production of neutralizing antibodies (IgG2a) against viral antigens requires the participation of CD4⁺ T-helper cells, which recognize antigen in association with MHC class II antigens, and an increased frequency of the HLA-DRB1*7 polymorphism has been observed in “at risk” nonresponders to influenza vaccination (Gelder et al., 2002).

The absence of a highly effective adjuvant constitutes a significant obstacle to the successful development of vaccines, particularly those directed against intracellular pathogens, requiring cellular immunity. Presently, there is an unmet need for an effective non-toxic composition or method capable of enhancing the antibody response to influenza and other vaccines.

In this connection, due to the properties evidenced above, cytokines, and in particular interferons (IFNs), have been considered in the art as possible adjuvants (Heat et al., 1992).

Interferons are multifunctional cytokines classified on the basis of structure:

i) Type I IFNs, encoded by genes devoid of introns and which include the IFN-α family, of at least 13 functional IFN-α subtypes, IFN-β and IFN-ω, produced effectively by all cell types.

ii) Type II IFN, encoded by a single intron-containing gene, also named IFN-γ, and produced primarily by T-cells and NK cells in response to specific antigen or mitogens.

Originally taken to be simple antiviral substances, type I IFNs have subsequently been shown to exhibit a variety of biological effects, including antitumor activities in experimental animal models as well as in patients.

Both Type I and Type II IFNs have been shown to exert potent inhibitory effects on antibody production and T cell proliferation in vitro, raising the question of whether these cytokines would act in a stimulatory or inhibitory manner in vivo. An ensemble of data obtained in different model systems have recently indicated the importance of type I IFN in the induction of a Th1 type of immune response and in supporting the proliferation, functional activity and survival of certain T cell subsets (Belardelli F. and Gresser I., 1996; and Tough et al., 1996).

Type I interferons are currently the most widely used cytokines in clinical practice. In particular, IFN-α is used worldwide in over 40 countries for the treatment of some viral diseases (especially Hepatitis C) and various types of human cancer, including some hematological malignancies (hairy cell leukemia, chronic myeloid leukemia, some B and T cell lymphomas) and certain solid tumors, such as melanoma, renal carcinoma and Kaposi's sarcoma. In contrast, IFN-γ has found limited clinical application, due at least in part to toxicity. Over the last few years, several studies have provided evidence that the biologic effects exerted by type I and type II IFNs can substantially differ in terms of type of activity in different experimental models. In some cases, such as melanoma and multiple sclerosis, the clinical use of IFN-γ has led to opposite effects with respect to those achieved with type I IFN.

In spite of its wide clinical use, type I IFN is not yet used as a vaccine adjuvant in man.

A relevant use of IFNs in vivo as adjuvants in vaccines has been shown for type II IFN (i.e., IFNγ). In particular, in EP 0241725, a vaccine is described containing a crude protein extract, derived from blood cells of mice infected with the virulent YM line of Plasmodium yoelii, which includes IFN-γ as an adjuvant. The amount of IFN-γ included in the vaccine is indicated in the range of 1,000 to 10,000 units per dose, wherein the amount of IFN-γ producing the adjuvant effect is indicated as 100 to 50,000 units. The dosage used is 5,000 units, even if doses lower than 200 units have been indicated also as effective.

The use of type I IFN as an adjuvant has been envisaged by some prior art documents, and type I IFN has been shown to enhance an in vivo protective Th1 type response when used as vaccine adjuvant.

IFN-α is a powerful polyclonal B-cell activator which induces a strong primary humoral immune response characterized by isotype switching and protection against virus challenge (LeBon et al., 2001). Indeed, IFN-α secreted by plasmacytoid dendritic cells in response to virus infection has been shown to induce B lymphocytes to differentiate into antibody producing plasma cells and to be necessary for the production of both specific and polyclonal IgGs in response to influenza infection (Jego, 2003). Furthermore, IFN-α has also been shown to stimulate the IgG2a antibody response characteristic of Th1 immunity and protection against virus infection (Le Bon et al., 2001) and has also been shown to be an unusually powerful adjuvant when mixed with influenza vaccine and injected intramuscularly (Proietti et al., 2002). In contrast, adjuvants such as alum potentiate IgG1 production characteristic of a Th2 response. IFN-α has also been shown to markedly enhance the proliferation of human tonsillar B-cells in response to anti-IgM antibodies. In addition, oromucosally administered IFN-α, when mixed and co-administered with human influenza vaccine, has also been shown to be an active mucosal adjuvant resulting in enhanced levels of secretory IgA (Proietti et al., 2002).

Type I interferons, predominantly interferon-α (IFN-α) and interferon-β (IFN-β), are produced at mucosal surfaces as part of the innate immune response to infectious agents. Oromucosal administration of recombinant IFN-α mimics oromucosal production of IFN and has been shown to confer protection against virus infection and tumor cell multiplication (Tovey and Maury, 1999). Protection occurs without toxicity through stimulation of cellular immunity in the absence of circulating levels of IFN (Eid et al., 1999). In particular, oromucosal administration of IFN-α stimulates both the maturation of dendritic cells and antigen presentation, and the T-helper type I (Th1) lymphocyte response to foreign antigens (Belardelli et al., 2002). Thus, oromucosal IFN therapy is most effective in stimulating host defenses during an ongoing immunologic reaction, whether in response to viral or tumor antigens.

U.S. Pat. Nos. 6,007,805 and 6,436,391 disclose the use of interferon-α subtypes as adjuvants in vaccine compositions, particularly anti-viral vaccine compositions.

There is also a need for effective adjuvants for use with anti-cancer vaccines. For example, although melanoma is one of the prototypic immunogenic tumors, the majority of tumor antigens are also self-antigens, limiting the therapeutic effectiveness of cancer vaccines due to tolerance to self-antigens. Thus, vaccines derived from melanoma antigens of the MAGE family, including tyosinase, Melan-A/MART-1, MAGE-A3/MAGE-A6, Trp-2, and gp100, result in only transient activation of cytotoxic T-cells and a limited clinical response even though a number of vaccination approaches have been used. These include direct immunization (peptide vaccines), viral vectors or naked DNA expressing the peptide, or loading antigen presenting cells such as dendritic cells with antigen (dendritic cell based vaccines).

Adoptive transfer of cells provides a means of overcoming tolerance by selection and activation of highly reactive T-cell sub-populations combined with lympho-depletion of T-regulatory cells. Thus, adoptive transfer of autologous tumor-reactive T-cells together with high-dose IL-2 therapy following non-myeloablative lymphodepleting chemotherapy (cyclophosphosphamide and fluarabine) resulted in the rapid growth in vivo of clonal populations of T-cells specific for the MART-1 melanocyte differentiation antigen and resulted in the destruction, of metastatic tumors and objective clinical responses in patients with progressive disease (Stage IV melanoma) refractory to standard therapy. Some patients with concomitant tumor regression also showed signs of autoimmune melanocyte destruction including vitiligo and anterior uveitis (Dudley et al., 2002). Addition of CpG oligonucleotides markedly potentiated the anti-tumor activity of adoptive transfer of autologous tumor-reactive T-cells together with high-dose IL-2 therapy following non-myeloablative lymphodepleting chemotherapy (Restifo, 2004).

Treatment of Stage IV melanoma with MAGE 3 protein vaccine together with 1.0 CpG resulted in 1 CR, 2PR, 2SD, and 2PD (Davis, 2004).

CpG oligonucleotides enhance both humoral and cell-mediated antigen-specific response to a wide range of antigens. Non-methylated CpG motifs are one of several pathogen associated molecular patterns (PAMPs) that activate the innate immune system via Toll-like receptors (TLR) present on the surface of antigen-presenting cells (APC). CpG activates TLR-9 which is found on the surface of human B-cells and plasmacytold dendritic cells (pDC) only. Toll-like receptors such as TLR-9 function as a bridge between the innate and adaptive immune response resulting in direct activation of B-cells and immunoglobulin production, activation of pDC, up-regulation of MHC class 11 antigens, B7 expression, and CD40 expression, and enhanced antigen presentation, and activation of CD4+ and CD8+ T-cells and a Th1 cytokine response. The activity of CpG oligonucleotides is dependent upon Type I IFN receptor signaling and addition of exogenous Type I IFN can bypass an upstream lesion in the pathway and can substitute for CpG and induce up-regulation of co-stimulatory molecules in the absence of any microbial stimulus. Furthermore, CpG oligonucleotides are devoid of adjuvant activity in IFN-α/β receptor−/−mice (IFNAR1−/−) or in normal mice treated with a polyclonal anti-IFN-α/β antibody (Le Bon, et al., 2001; and Proietti et al., 2002).

Accordingly, despite the availability of potentially efficacious recombinant antigens, weakness or absence of responsiveness to vaccination and patient compliance still remains the major concerns for prophylactic or therapeutic subunit vaccines. The weak immunogenicity of subunit vaccines makes it necessary for these vaccines to be given multiple times in order to elicit a satisfactory response, making lack of patient compliance a significant problem.

Therefore, a composition that improves antigen immunogenicity and promotes consistently strong immune responses, lowering the number of vaccine doses required to induce seroconversion/seroprotection, even to a single dose, would find widespread application.

Citation of any document herein is not intended as an admission that such document is pertinent prior art, or considered material to the patentability of any claim of the present application. Any statement as to content or a date of any document is based on the information available to applicant at the time of filing and does not constitute an admission as to the correctness of such a statement.

### SUMMARY OF THE INVENTION

The present invention relates to a method of enhancing the immune response to a vaccine by oromucosally administering substantially concurrently with the administration of the vaccine, an amount of interferon and/or other Th1-stimulatory cytokines, sufficient to enhance the immune response to the vaccine. The immune response that is being enhanced may be either or both of a humoral or a cellular response. The present invention is particularly effective in enhancing Th1 type humoral immunoresponse to a vaccine in a protective immunization treatment.

The oromucosally administered interferon and/or other cytokines, effectively serves as an immunoadjuvant when administered substantially concurrently with an administration of a vaccine, because it enhances the antibody and cellular immune response to the vaccine. The enhanced immune response is characterized by long-term antibody production and immunological memory. As the oromucosal administration of interferon, and/or other cytokine, does not involve transfer of interferon and/or other cytokine to the bloodstream, large amounts of interferon and/or other cytokine may safely be used without eliciting a toxic response. This is a great improvement over the use of known and currently available adjuvants, such as alum.

### BRIEF DESCRIPTION OF THE DRAWINGS

FIG. 1A shows the effect of IFN-α on the anti-influenza antibody response 15 days after vaccination. Antibody response was determined using antigen capture ELISA assays for the specific immunoglobulin sub-classes: total IgG, IgG1, IgG2a, and IgA as indicted in the Figure. Each antibody sub-class was measured after im administration of VAXIGRIP™ anti-influenza vaccine either alone, or admixed with 10⁵ IU of recombinant IFN-α or with concurrent administration of 10⁵ IU of recombinant IFN-α om.

FIG. 1B shows the effect of IFN-α on the anti-influenza antibody response 30 days after vaccination. Antibody response was determined using antigen capture ELISA assays for the specific immunoglobulin sub-classes: total IgG, IgG1, IgG2a, and IgA as indicted in the Figure. Each antibody sub-class was measured after im administration of VAXIGRIP™ anti-influenza vaccine either alone, or admixed with 10⁵ IU of recombinant IFN-α or with concurrent administration of 10⁵ IU of recombinant IFN-α om.

FIG. 2A shows the effect of IFN-α on the anti-influenza antibody response after revaccination. Mice were vaccinated on day 0 by im injection of 15 micrograms of VAXIGRIP™ anti-influenza vaccine either alone, or admixed with 10⁵ IU of recombinant IFN-α or with concurrent administration of 10⁵ IU of recombinant IFN-α om. At 90 days, animals were revaccinated by im injection with 15 micrograms of VAXIGRIP™ either alone, or admixed with 10⁵ IU of IFN-α, or with concurrent administration of 10⁵ IU of IFN-α om. The anti-ifluenza antibody response was determined at 10⁵ days using antigen capture ELISA assays for the specific immunoglobulin sub-classes: total IgG, IgG1, IgG2a, and IgA as indicated in the Figure.

FIG. 2B shows the effect of IFN-α on the anti-influenza antibody response after revaccination. Mice were vaccinated on day 0 by im injection of 15 micrograms of VAXIGRIP™ anti-influenza vaccine either alone, or admixed with 10⁵ IU of recombinant IFN-α or with concurrent administration of 10⁵ IU of recombinant IFN-α om. At 90 days, animals were revaccinated by im injection with 15 micrograms of VAXIGRIP™ either alone, or admixed with 10⁵ IU of IFN-α, or with concurrent administration of 10⁵ IU of IFN-α om. The anti-ifluenza antibody response was determined at 120 days using antigen capture ELISA assays for the specific immunoglobulin sub-classes: total IgG, IgG1, IgG2a, and IgA as indicated in the Figure.

FIG. 2C shows the effect of IFN-α on the anti-influenza secretory s-IgA antibody response after revaccination. Mice were vaccinated on day 0 by im injection of 15 micrograms of VAXIGRIP™ anti-influenza vaccine either alone, or admixed with 10⁵ IU of recombinant IFN-α or with concurrent administration of 10⁵ IU of recombinant IFN-60 om. At 90 days, animals were revaccinated by im injection with 15 micrograms of VAXIGRIP™ either alone, or admixed with 10⁵ IU of IFN-α, or with concurrent administration of 10⁵ IU of IFN-α om. The secretory s-IgA anti-ifluenza antibody response was determined at 120 days using an antigen capture ELISA assay specific for secretory s-IgA as indicated in the Figure.

### DETAILED DESCRIPTION OF THE INVENTION

The present invention is based on the surprising discovery that treatment of animals with recombinant interferon-α (IFN-α) by the oromucosal route markedly enhances the humoral response to the distant intramuscular injection (im) of commercially available influenza vaccine (VAXIGRIP™, Avantis Pasteur MSD). Indeed, all four classes of influenza specific immunoglobulins tested (total IgG, IgG1, IgG2a, and IgA) were found to be markedly increased in response to influenza vaccination following oromucosal (om) administration of IFN-α in a dose dependent manner. Even more remarkably, the immunoadjuvant effect of oromucosally administered IFN-α was under certain circumstances greater than that obtained when IFN-α was mixed with influenza vaccine and the mixture injected intramuscularly (im).

These results may be explained in part by the results of previous studies showing that oromucosal administration of IFN-60 results in a rapid migration of immunocompetent cells from the peripheral lymph nodes to the site of systemically injected antigen (whether tumor or viral antigen), due to the rapid induction of chemokines such as Crg2 (which regulate lymphocyte trafficking), in the absence of systemic absorbance of the IFN protein. In contrast, parenteral injection of IFN-α at a site distant from the site of vaccination most probably induces migration of dendritic cells and other antigen presenting cells towards the site of IFN injection and away from the vaccination site, thus reducing the humoral response to the influenza vaccine.

This is the first demonstration that a substance, herein referred to as an “immunoadjuvant”, can enhance the immune response to a vaccine when administered at a site distant from the site of vaccination. The term “immunoadjuvant” is used as the interferon is not acting as a typical adjuvant, which is a substance that must be mixed with the vaccine in order to enhance the antigenicity of the vaccine. On the other hand, the function of the interferon in the present invention is not merely due to its immunostimulatory effects, as disclosed, for example, in U.S. Pat. Nos. 6,361,769 and 6,660,258 and U.S. patent publication 2003/0108519. These previous patent disclosures relating to the immunostimulatory effect of oromucosally administered interferon for the treatment of viruses, tumors, and other pathogens, rely on a rapid cellular immunoresponse. Thus, the therapeutic effects observed occurred prior to the establishment of an effective antibody response, which consequently was never studied and, in any event, could not have been involved in many of the experiments reported therein. Indeed, whether interferons would be expected to exert a stimulatory or an inhibitory effect on antibody production has been an open question. Therefore, the immunoadjuvant effect reported herein is different from, and would not have been expected from, the previous reports of the immunostimulatory effects of interferon or the adjuvant effects of interferon. So as to distinguish the present invention from the embodiment of Proietti, 2002, involving the oromucosal administration of a vaccine mixed with IFN-α as an adjuvant, the present invention hereby explicitly excludes use of om IFN in conjunction with oromucosally administered vaccine.

A single oromucosally administered interferon containing formulation, such as a tablet or lozenge, taken at the time of vaccination, markedly increases the antibody response, and hence the degree of protection, not only for influenza vaccine but it would be expected to be applicable for all vaccines. A distinct advantage of an oromucosal IFN formulation is that it can be used to improve the protective effect of a particular vaccine that has been approved by an appropriate regulatory agency, without the need to re-register that vaccine with that agency, as would be the case for a novel adjuvant that must be administered in admixture with a vaccine.

Numerous studies have shown that the mucosal immunity acquired by natural influenza virus infection, which is due to the presence of secretory s-IgA in the respiratory tract, is more effective in preventing infection, particularly against variant virus infections, than systemic immunity due to serum IgG induced by parenteral vaccination. See Ito et al., 2003. Serum IgG in the immunized individual appears to be important for preventing lethal pneumonia rather than protecting against infection. Oromucosal administration of IFN-α increases serum IgA anti-influenza antibodies to a similar or greater extent as when IFN-60 is mixed with the vaccine and the mixture injected intramuscularly. A similar effect was also observed on anti-influenza s-IgA antibody production in the lungs following oromucosal administration of IFN-α. Experimental results further indicate that oromucosal IFN-α also reduces the time required to attain a maximal antibody response and hence full protection following vaccination.

The immune response induced by Type I IFN-or IFN-α is a Th1 type response characterized by a specific Ig profile, namely, in mice, by the specific induction of circulating IgG2a and/or secretory IgA, which confers protection from pathogen challenge such as bacteria or viruses.

In the non-toxic immunoadjuvant composition of the present invention, the IFN can be any interferon that belongs to the Type I IFN family, or it can be IFN-γ, sometimes referred to as Type II IFN. In this connection, the most effective dosage in humans is in the range of 10⁵-10⁸ IU, preferably 10⁶-10⁷ IU.

The following are non-limiting examples of sources of IFN that can be used in the present invention: natural IFN-α (a mixture of different IFN-α subtypes or individual IFN-α subtypes) from stimulated leukocytes of healthy donors or lymphoblastoid IFN-α from Namalwa cells: a synthetic type I IFN, such as consensus IFN (CIFN); recombinant IFN-β (commercially available as REBIF™, Serono; AVONEX™, Biogen; and BETASERON™, Berlex) or recombinant IFN-α subtypes, such as IFN-α2a (commercially available as ROFERON™, Roche) and IFN-α2b (commercially available as INRON-A™, Schering Plough), or IFN-ω; new IFN molecules generated by the DNA shuffling method or site-directed mutagenesis, provided that they are used in the above mentioned dosages indicated per vaccine dose; and recombinant human IFN-α or IFN-β molecules having one or more amino acid substitutions, deletions or additions or otherwise obtained with naturally occurring polymorphisms such as the polypeptides of GenOdyssee, WO 02/101048 (see WO 02/083733, WO 03/000896, and others).

In the pharmaceutical composition for the oromucosally administered interferon dosage form of the present invention, a variety of vehicles and excipients for IFN may be used, as will be apparent to the skilled artisan. Representative formulation technology is taught in, inter alia, Remington 1995, and its predecessor editions. The IFN formulation may comprise stability enhancers, such as glycine or alanine, as described in U.S. Pat. No. 4,496,537, and/or one or more carriers, such as a carrier protein. For example, for treatment of humans, pharmaceutical grade human serum albumin, optionally together with phosphate-buffered saline as diluent, is commonly used. Where the excipient for IFN is human serum albumin, the human serum albumin may be derived from human serum, or may be of recombinant origin. Normally when serum albumin is used it will be of homologous origin.

The IFN may be administered by any means which provides contact of the IFN with the oromucosal cavity of the recipient for a sufficient time to allow the interferon to effectively enhance the immune response to the concurrently administered vaccine. This requires contact of the interferon with the oromucosa for a period of at least about 5 seconds, preferably 1-2 minutes, and possibly as long as 5 minutes, i.e., 5-360 seconds. Thus, oromucosal administration is definitely distinguishable from oral administration. If a tablet or liquid composition for oral administration is merely swallowed, there will not be sufficient time of contact of the interferon with the oromucosa to permit the enhancement of the immune response to the vaccine.

Within these parameters, it will be clearly understood that the IFN dosage form used in the method of the present invention is not limited to any particular type of formulation. The IFN may be administered deep into the oromucosal cavity; this may be achieved with liquids, solids, or aerosols, as well as nasal drops or sprays. Thus, the formulation includes, but is not limited to, liquid, spray, syrup, lozenges, buccal or sublingual tablets, and nebuliser formulations. A person skilled in the art will recognize that for aerosol or nebuliser formulations the particle size of the preparation may be important, and will be aware of suitable methods by which particle size may be modified. Thus, for aerosol or formulations, the particle size must be so large as to allow deposition of the interferon in the nasopharyngeal mucosa, such that it may remain there for the requisite period of time. Formulations intended for administration to the lungs are not considered to be oromucosal formulations, as the particle size will be so small as to bypass deposition on the nasopharyngeal mucosa and to travel directly into the lungs, which is its desired site of action and where it is taken up into the circulation.

Representative formulations of interferon for oromucosal use include the following (all % are w/w):

Tablet: Dextrose BP 45%; gelatin BP 30%; wheat starch BP 11%; carmellose sodium BP 5%; egg albumin BPC 4%; leucine USP 3%; propylene glycol BP 2%; and 5×10⁶ IU IFN-α2. The tablet may be used as is and allowed to slowly dissolve in the mouth or may be dissolved in water and held in the mouth as needed.

An interferon paste may be prepared, as described in U.S. Pat. No. 4,675,184, from glycerin 45%, sodium CMC 2%, citrate buffer (pH 4.5) 25%, distilled water to 100%, and 5×10⁶ IU IFN-α2. The interferon paste may be adhered to the buccal mucosa.

Likewise, a gargle or syrup may be prepared by adding the desired amount of interferon to a commercially available mouthwash or cough syrup formulation.

In the animal experiments described in this specification, the oromucosal administration is achieved by administration of the IFN preparation deep into the nasal cavity, so that it is rapidly distributed into the oropharyngeal cavity, i.e., the mouth and throat of the recipient mammal, so as to make contact with the mucosa lining this cavity.

It is known from previous work from the laboratory of the present inventor that oromucosal administration of interferon does not result in the appearance of circulating levels of IFN (Eid et al., 1999 and U.S. Pat. No. 6,361,769).

While the thrust of the present description relates to interferon as the immunoadjuvant, the present invention is also extended to the use of other cytokines that are known to play a role in antibody production and/or stimulate Th1 response. U.S. Pat. No. 6,660,258, to the present inventor, is directed to the oromucosal administration of any Th1 or Th2 specific cytokine at doses that induce a host defense mechanism stimulating effect. Among the Th1 cytokines disclosed therein, besides interferons, are IL-2, IL-12, IL-15, IL-18, granulocyte macrophage-colony stimulating factor (GM-CSF), and tumor necrosis factor beta (TNF-β or lymphotoxin). Thus, these cytokines, either alone or in combination with interferon and/or one another, may also be used as the immunoadjuvant of the present invention in the same manner as described herein for interferon, mutatus mutandi. Thus, the effective dose for each can readily be determined by one of ordinary skill in the art without undue experimentation.

Antigens include purified or partially-purified preparations of protein, peptide, carbohydrate or lipid antigens, and/or antigens associated with whole cells, particularly dendritic cells that have been mixed with the antigen. On the whole, any pathogen or tumor and/or differentiation associated antigen can be considered as a possible immunogen to be given at the same time as the IFN as immunoadjuvant, and can be easily identified by a person skilled in the art.

While the experiments in the present specification relate to the influenza vaccine VAXIGRIP™, it is fully expected that the present invention will enhance the immune response to the administration of any vaccine. While the term “vaccine” is often used to refer only to vaccinations intended to induce prophylaxis, the term as used throughout the present specification and claims is intended to include vaccination for therapeutic purposes as well. For example, vaccines that comprise tumor-associated antigens are intended to induce an immune response against tumors. Vaccines to viral particles may be used not only to create prophylaxis against the virus, but also to eradicate an existing viral infection. Thus, for example, vaccines are available against HBV and others against AIDS and HCV, which are in active development. Active vaccination against amyloid-β plaques are also in development for the treatment of Alzheimer's disease. Thus, the term “vaccine” applies to the administration of any antigen for the purpose of inducing an immune response against that antigen or to a cross-reactive antigen that exists in situ. Preferred vaccines include an influenza, smallpox, anthrax, hepatitis B virus, human pappilloma virus, herpes simplex virus, polio, tuberculosis or anti-cancer vaccine.

Among the reasons why it would expected that the present invention would be applicable to any such vaccine in light of the results shown for influenza vaccine, is the fact that interferons are already known to be effective as an adjuvant when administered in admixture. See, for example, Proietti, 2002, WO 02/083170 and Le Bon et al., 2001. Furthermore, once it is established that oromucosally administered interferon enhances the immune response to an antigen which is remotely but concurrently administered, there is no reason to believe that the same will not be true when the subject is vaccinated with any antigen that causes an immune response. The known effects of oromucosally administered interferon for therapeutics (see U.S. Pat. No. 6,361,769, for example), combined with the known effects of interferon as an adjuvant, would permit one to reasonably extrapolate the results which have been shown for influenza vaccine to any other vaccine.

It is known that existing adjuvants intended to improve antigenicity of a vaccine, such as alum, have severe side effects. While the vaccine used in the present invention may include an adjuvant in its composition, it would be desirable to be able to eliminate such adjuvants and still have a vaccine with a satisfactory immune response. It is expected that the use of the oromucosally administered interferon immunoadjuvant will serve this purpose. However, to the extent that any vaccine might have no measurable immunological response without an adjuvant, the effect of the immunoadjuvant of the present invention would have to be tested on a case by case basis.

The amount of antigen(s) present in each vaccine dose, adjuvanted or not, is selected as an amount capable of inducing a protective immune response in vaccinated subjects. This amount will depend on the specific antigen and the possible presence of typical adjuvants, and can be identified by a person skilled in the art. In general, each dose will contain 1-1000 micrograms of antigen, preferentially 10-200 μg. Further components can be also present advantageously in the vaccine or in the adjuvanted-vaccine.

In some cases, the vaccine or the adjuvanted-vaccine can be injected subcutaneously or intramuscularly on the account of the expected effect and ease of use. Intradermal injection can effectively be performed for some vaccines and other delivery systems suitable for recruiting a relevant number of dendritic cells to the injection site could be considered. However, oromucosal administration is excluded.

Intranasal administration to the lungs and oral administration of the vaccine are also included especially for those infectious agents transmitted through these routes of infection such as viral respiratory infections, for example, influenza virus infection.

Moreover, intranasal, oral or any other mucosal administration of the vaccine or directly adjuvanted-vaccine also represents a valuable choice, which results in the induction of a potent protective local and/or systemic immunity by using a very practical modality of vaccine delivery.

A person skilled in the art can determine in this connection the most appropriate formulation as a function of the antigen the vaccination is directed to counteract.

The vaccine composition can be formulated in any conventional manner, as a pharmaceutical composition comprising sterile physiologically compatible carriers such as saline solution, excipients, adjuvants (if any), preservatives, stabilizers, etc.

The vaccine can be in a liquid or in lyophilized form, for dissolution in a sterile carrier prior to use. The presence of alum or liposome-like particles in the formulation are also possible, since they are useful for obtaining a slow release of the antigen(s). Other strategies for allowing a slow release of the vaccine can be easily identified by those skilled in the art and are included in the scope of this invention.

The pharmaceutically acceptable carrier vehicle or auxiliary agent can be easily identified accordingly for each formulation by a person skilled in the art.

The method of the present invention can be used in both prophylactic and therapeutic treatment of infectious diseases and cancer. In particular, the method of the present invention can be used in a treatment for preventing viral and bacterial diseases (i.e., prophylactic vaccines) as well as for the treatment of severe chronic infection diseases (i.e., therapeutic vaccines). Moreover, the method can also be used in the prevention and treatment of cancer or other diseases and conditions when suitable antigens are used.

This can be achieved by using antigens against infectious agents associated with human malignancies, e.g., EBV, HPV and H. pilori, or well defined tumor associated antigens such as those characterized in human melanoma, e.g., MAGE antigens, thyrosinase gap 100, and MART, as well as in other human tumors.

In particular, the method of the present invention is particularly suitable for vaccination of the so-called low-or non-responder subjects, such as immuno-compromised subjects like maintenance hemodialysis, transplanted and AIDS patients. In general, the method of the present invention is advantageously suitable for vaccination of individuals at high risk of infection in any situation for which an earlier seroconversion/seroprotection is desirable.

These characteristics are in particular referred to vaccination against HBV.

As an additional example, the method of the present invention can be particularly valuable for inducing protection against influenza virus in elderly individuals poorly responsive to standard vaccination.

For the HBV vaccine as well as for other viral vaccines, the s.c. or intramuscular route of injection can be preferable, while in other cases intranasal administration into the lungs can exhibit advantages in terms or efficacy and/or patient compliance, especially for agents capable of infecting the host through the respiratory system.

The method of the present invention may be used even when the vaccine is administered orally. Oral administration of a vaccine involves swallowing of the vaccine, and therefore does not include oromucosal delivery, which requires at least 5 seconds of contact time with the oromucosa. Accordingly, if the vaccine is to be administered orally, the interferon and/or other Th1 stimulating cytokine may still be administered oromucosally within a short time before or after the oral administration of the vaccine. The same is true with respect to oromucosal administration of the interferon and/or other Th1 stimulating cytokine substantially concurrently with intranasal delivery of a vaccine to the lungs.

The immunoadjuvant of the present invention may be used both in conjunction with primary vaccination as well as with revaccination. Thus, the immune response against the antigen may be enhanced at any time that the subject is exposed to the antigen, including a revaccination as well as exposure after vaccination to the antigen against which the subject was vaccinated. Accordingly, the administration of the immunoadjuvant may also take place at the time that the subject is exposed to the antigen in question. For example, if a person is immunized against anthrax for protection against a possible bioterrorist attack, and sometime later that subject is exposed to the presence of anthrax in such an attack, the administration of the immunoadjuvant of the present invention would enhance the protective recall immune response against that antigen. The recall response is that responsive to activation of the memory cells in circulation following vaccination. Furthermore, because interferon is a polyclonal B cell activator, it would be expected that not only will the immune response protect the subject against the specific antigen against which he or she was vaccinated, but also would exhibit a degree of cross-protection against related and perhaps mutated antigens to which that subject might be exposed in the future. This would be particularly important for protection against influenza, which is known to exhibit both antigenic shift and drift.

Accordingly, another aspect of the present invention is the treatment of an infection or other exposure to an antigen against which the subject was previously vaccinated by oromucosally administering a recall response enhancing amount of interferon as an immunoadjuvant.

Having now generally described the invention, the same will be more readily understood through reference to the following example which is provided by way of illustration and is not intended to be limiting of the present invention.

### EXAMPLE

### Example 1

### Effect of Oromucosally Administered IFN-α on the Anti-Influenza Antibody Response to an Influenza Vaccine on Primary Vaccination

Groups of ten 6-8 week old male C57B1/6 were treated with 15 micrograms of VAXIGRIP™ (Aventis Pasteur MSD) at day 0 by intramuscular (im) administration, either alone or mixed with an equal volume of PBS containing increasing quantities (10³, 10⁴, 10⁵, 10⁶, 10⁷ IU) of recombinant mouse IFN-α or recombinant human IFN-α, or PBS containing a quantity of BSA that is equivalent to the quantity of interferon used. Other groups of mice received the vaccine via intramuscular (im) injection and increasing amounts of interferon by the oromucosal (om) route on either day −2, −1, 0, +1 or +2, relative to the im vaccination of the animals. Other groups of animals were treated with IFN or BSA alone, either by im injection or by the om route, and left unvaccinated.

Antibody response was determined at 15 and 30 days using antigen capture ELISA assays specific for the following immunoglobulin subtypes: total IgG, IgG1, IgG2a, and IgA in the serum, and secretory IgA in the lungs.

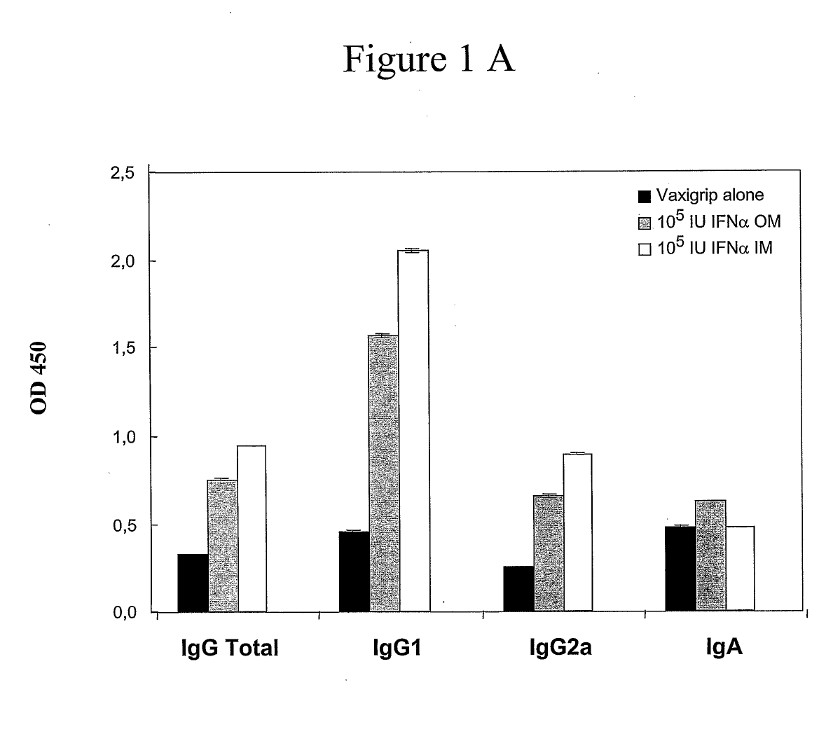

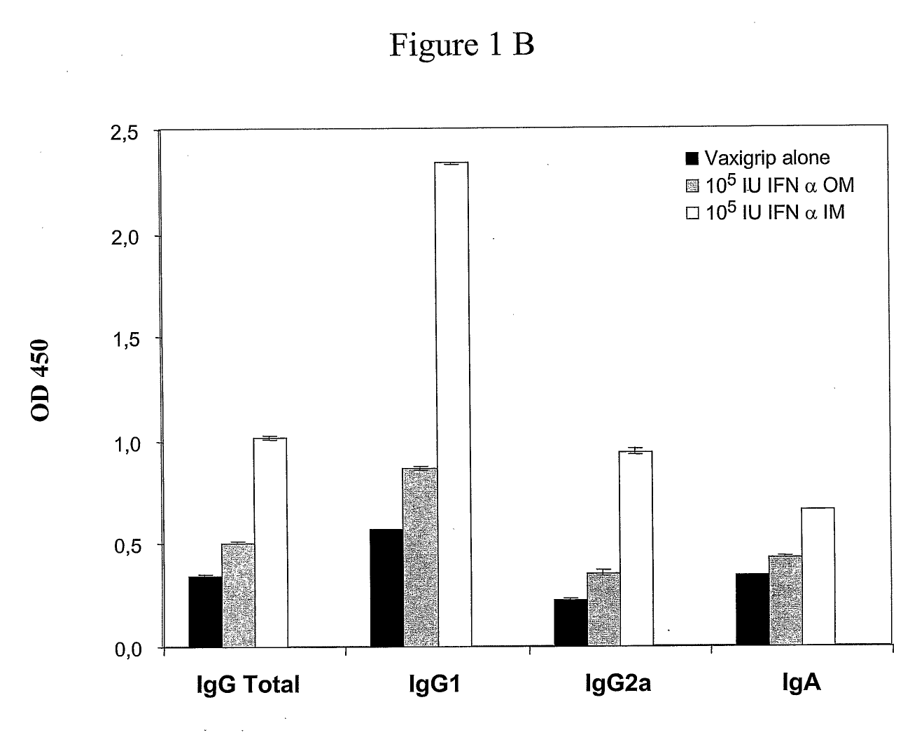

The results are shown in FIG. 1A and FIG. 1B. FIG. 1A shows the effect of IFN-α on the anti-influenza antibody response after 15 days. For each of the antibody types measured, the column on the left is after im administration of the vaccine alone without concurrent administration of interferon, the middle column is after im administration of the vaccine with concurrent om administration of IFN, and the column on the right is after im administration of a mixture of the vaccine and IFN. FIG. 1B shows the effect of IFN-α on the anti-influenza antibody response measured after 30 days. It can be seen in every case that the antibody response when the vaccine is administered with om interferon is more pronounced than the response obtained without the interferon.

While administration of the interferon intramuscularly mixed with the vaccine in most cases gives a somewhat better result than the om administered interferon. There are still substantial advantages to administering the interferon sublingually as opposed to im. First, there are often significant side effects to the administration of high doses of interferon im. Secondly, the use of interferon as an immunoadjuvant that is administered separately from the vaccine but substantially concurrently in time requires only registration for the interferon-containing formulation. In other words, using om interferon as an immunoadjuvant would likely not require separate re-registration of an already approved vaccine. When the interferon is an adjuvant that is mixed with the vaccine, as has been previously known in the art, then seperate registration of the vaccine/adjuvant mixture must be obtained, which is extremely expensive and time consuming.

It is generally known in the vaccine art that the peak of IgG antibody response to a vaccine appears at approximately 30 days after primary vaccination. It can be seen from comparing FIGS. 1A and 1B that there is a certain accelerating affect due to the om interferon administration.

The dose response curves (not shown) establish that the optimum response is obtained when the maximum amount of the interferon is administered (10⁵ in this particular experiment). In other tests using human interferon in mice, the dose response curve showed that the antibody response increases to a peak at a certain level and then decreases. Thus, it is expected that there will be an optimum amount of interferon for use as an immunoadjuvant, and that this amount can be determined empirically upon conducting of human testing.

Table 1 shows the results of a similar experiment after 15 days with the immunoglobulin titer expressed as an endpoint titer rather than optical density at a fixed serum dilution. This experiment also confirms that administration of IFN om provides substantially better results than the vaccine alone. However, this experiment shows that the results with om interferon are substantially better than the results with the mixed administration of im interferon and vaccine.

TABLE 1

| | Immunoglobulin: | | |
|---|---|---|---|
| | End Point Titers | | |
| Treatment | IgG | IgG1 | IgG2a |
| VAXIGRIP ™ | 102,400 | 102,400 | 102,400 |
| IFN-α 10⁵ IU OM | 409,600 | 409,600 | 409,600 |
| IFN-α 10⁵ IU IM | 204,800 | 204,800 | 204,800 |

A comparison of the experiments in which interferon was administered to mice at −2, −1, 0, +1, and +2 days (not shown), establishes that the optimum effect is obtained when the interferon is administered at day 0. When administered at day −1 or −2 by the om route, the desired effect does not occur. When administered on day +1 or +2 by the om route, the results are substantially the same as if the interferon had not been administered at all. Thus, it is apparent that the optimum time of IFN administration is substantially simultaneous with administration of the vaccine, which means within a few hours before or after such vaccine administration.

### Example 2

### Effect of Oromucosally Administered IFN-60 on the Anti-Influenza Antibody Response to an Influenza Vaccine on Secondary Vaccination

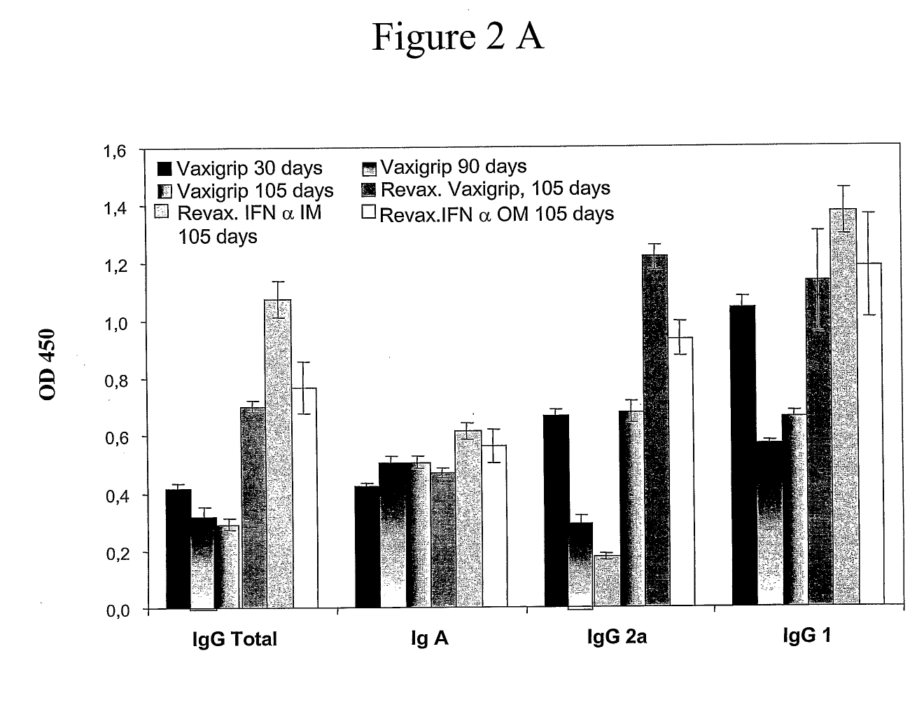

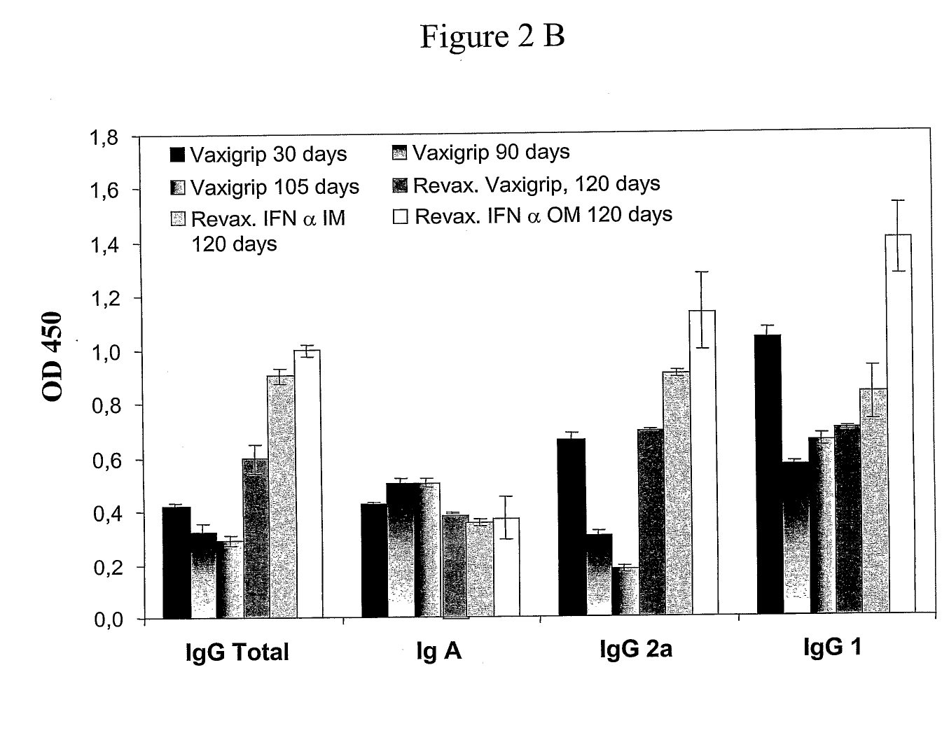

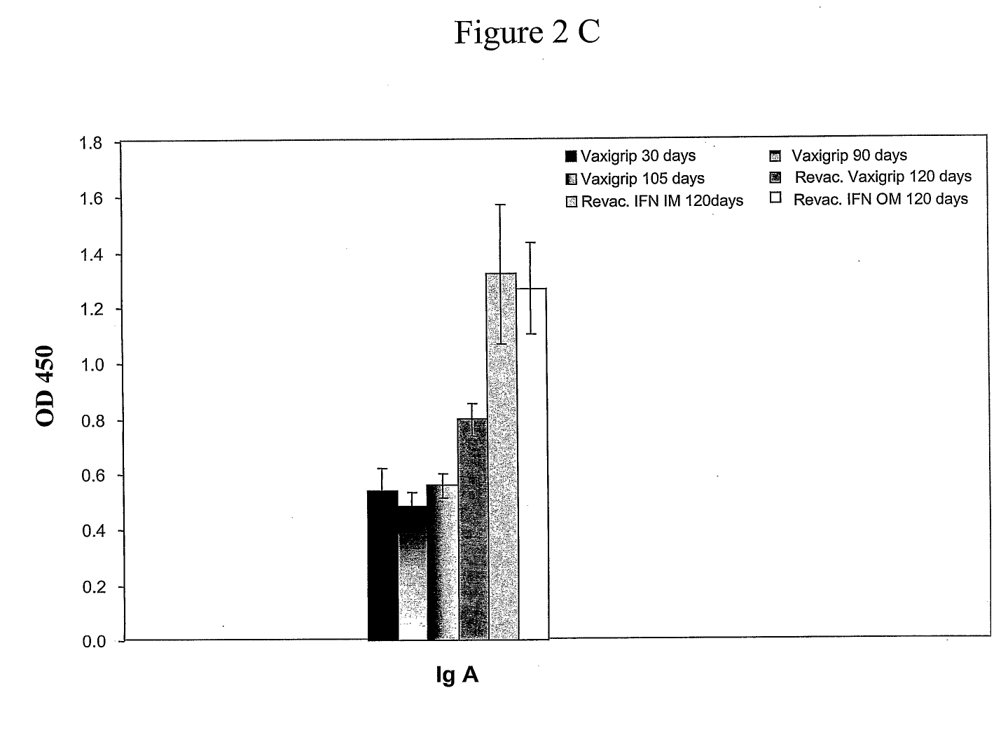

In experiment 2, the groups of mice were administered in the same manner as in experiment 1, except that on day 90, the mice were revaccinated. Each vaccination step, i.e., both the primary and the revaccination step, were done either with VAXIGRIP™ alone, VAXIGRIP™ mixed with IFN-α, or VAXIGRIP™ with concomitant om IFN-60 administration. FIGS. 2A, 2B and 2C show the results of these experiments, measured either after 105 days for FIG. 2A, or 120 days after initial vaccination in FIGS. 2B and 2C. The Ig titers are expressed as optical density determinations at a particular serum dilution. The optical density was measured at 450 nm. It can be seen that already at 15 days following revaccination, there is some increase in antibody titer in the mice administered with IFN om, particularly for the IgG2a, which is the Ig sub-type that is most important for protection. After 30 days (FIG. 2B), it is seen that there is a very significant effect with the om administered interferon. Indeed, the effect appears to be even higher than that obtained when the interferon is mixed with the vaccine and intramuscularly administered. Similarly, after 30 days (FIG. 2C), it is seen that there is also a very significant stimulatory effect with the om administered interferon an anti-influenza secretory s-IgA production in the broncho-alveolar lavage.

### Example 3

### Human Clinical Trials

To test the effect of the immunoadjuvant on humans, the following clinical tests will be conducted. In a population of 140 subjects, randomized into 2 groups, one group will be treated with interferon oromucosally, and then immediately vaccinated with VAXIGRIP™ intramuscularly, and the other group will be treated with a placebo om and then immediately vaccinated with VAXIGRIP™ im. Interferon will be administered by the oromucosal route in a dose of 5 ml of saline solution containing 5 million units of ROFERON™ recombinant IFN-α. The subjects will be instructed to maintain the saline in their mouth for two minutes before swallowing. The control subjects will receive saline only followed by vaccination with VAXIGRIP™ im. The subjects are aged 65-85, having no leukemia or solid tumors, or autoimmune disease, and with intact tonsils. The subjects have all received an influenza vaccine in the previous five years. The antibody response will be measured at 21 days, both by inhibition of hemagglutinin and by antigen capture ELISA for determination of the immunoglobulin sub-classes. Secretory IgA levels in saliva will also be measured. It is expected that the results will be comparable to those obtained in the pre-clinical animal experiments quoted in examples 1 and 2.

Having now fully described this invention, it will be appreciated by those skilled in the art that the same can be performed within a wide range of equivalent parameters, concentrations, and conditions without departing from the spirit and scope of the invention and without undue experimentation.

While this invention has been described in connection with specific embodiments thereof, it will be understood that it is capable of further modifications. This application is intended to cover any variations, uses, or adaptations of the inventions following, in general, the principles of the invention and including such departures from the present disclosure as come within known or customary practice within the art to which the invention pertains and as may be applied to the essential features hereinbefore set forth as follows in the scope of the appended claims.

All references cited herein, including journal articles or abstracts, published or corresponding U.S. or foreign patent applications, issued U.S. or foreign patents, or any other references, are entirely incorporated by reference herein, including all data, tables, figures, and text presented in the cited references. Additionally, the entire contents of the references cited within the references cited herein are also entirely incorporated by references.

Reference to known method steps, conventional methods steps, known methods or conventional methods is not in any way an admission that any aspect, description or embodiment of the present invention is disclosed, taught or suggested in the relevant art.

The foregoing description of the specific embodiments will so fully reveal the general nature of the invention that others can, by applying knowledge within the skill of the art (including the contents of the references cited herein), readily modify and/or adapt for various applications such specific embodiments, without undue experimentation, without departing from the general concept of the present invention. Therefore, such adaptations and modifications are intended to be within the meaning and range of equivalents of the disclosed embodiments, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance presented herein, in combination with the knowledge of one of ordinary skill in the art.

### REFERENCES

Belardelli F. and Gresser I. The neglected role of type I interferon in the T-cell response: implication for its clinical use. Immunol Today 17: 369-372, 1996
Davis, H., Coley Pharmaceuticles, Ottawa Canada, Keystone Symposium, Rational Design of Vaccines and Immunotherapeutics, Keystone, 6-11 Jan. 2004
Dudley et al., Science, 298, 850-854, 2002
Eid et al., J. IFN and Cytokine Res., 1999, 19, 157-169
Gelder, et al., J. Inf. Dis., 185, 114-117, 2002
Jego, et al., Immunity, 2003, 19, 225-234.
Le Bon, et al., Immunity, 14, 461-473, 2001
Lien and Golenbock in Nature Immunology, 4, 1162-1164, 2003
Oxford et al., Vaccine, 21, 2743-2746, 2003
Proietti et al, J. Immunol, 2002, 169, 375-383
Remington: The Science and Practice of Pharmacy, 189^{th} ed., Mack Publishing Co., Easton, Pa., 1995
Restifo, N., NCI, Keystone Symposium, Rational Design of Vaccines and Immunotherapeutics, Keystone, 6-11 Jan. 2004.
Spraycar, Marjory, ed., Stedman's Medical Dictionary, Williams and Wilkins, 1995
Tough D F, Borrow P, and Sprent J. Induction of bystander T cell proliferation by viruses and type I interferon in vivo. Science 272: 1947-1950, 1996
Tovey and Maury, J. IFN and Cytokine Res., 1999, 19, 145-155

What is claimed is:

1. A method of enhancing immune response to a vaccine, comprising:
(a) administering an effective amount of a vaccine or antigen to a subject by a means other than oromucosal delivery; and
(b) oromucosally administering an amount of interferon and/or at least one other Th1-stimulating cytokine sufficient to enhance the immune response to the vaccine, said interferon and/or other cytokine administration being substantially concurrent with said vaccine administration.

2. A method in accordance with claim 1, wherein said immune response is a humoral response.

3. A method in accordance with claim 1, wherein said immune response is a cellular response.

4. A method in accordance with claim 1, wherein said vaccine is administered intramuscularly.

5. A method in accordance with claim 1, wherein said vaccine is administered orally or intranasally into the lungs.

6. A method in accordance with claim 1, wherein said vaccine is administered subcutaneously or intradermally.

7. A method in accordance with claim 1, wherein said interferon is administered in a manner such that the interferon is maintained in contact with the oral mucosa for at least 5 seconds.

8. A method in accordance with claim 1, wherein said interferon is administered in a manner such that the interferon is maintained in contact with the oral mucosa for at least one minute.

9. A method in accordance with claim 1, wherein said interferon is administered in a manner such that the interferon is maintained in contact with the oral mucosa from 5-300 seconds.

10. A method in accordance with claim 1, wherein said vaccine is administered without the presence of an adjuvant.

11. A method in accordance with claim 1, wherein said vaccine is an adjuvanted vaccine.

12. A method in accordance with claim 1, wherein said vaccine is an influenza, smallpox, anthrax, hepatitis B virus, human pappilloma virus, herpes simplex virus, polio, tuberculosis or anti-cancer vaccine.

13. A method in accordance with claim 1, wherein said effective amount of interferon is within the range of 10⁵-10⁸ IU.

14. A method in accordance with claim 1, wherein said effective amount of interferon is within the range of 10⁶-10⁷ IU.

15. A method in accordance with claim 1, wherein said interferon is selected from the group consisting of a Type I interferon or IFN-γ.

16. A method of enhancing the immune response in a subject upon exposure to an antigen to which the subject has been previously immunized, comprising, upon exposure to said antigen, oromucosally administering interferon, and/or one or more other Th1 stimulatory cytokines, in an amount sufficient to enhance the recall response to said antigen.
